(19) United States
(12) Patent Application Publication
(10) Pub. No.: US 2006/0113190 A1
(43) Pub. Date: Jun. 1, 2006

(54) Microfluidic device and method for improved sample handling

(76) Inventor: Ronald T. Kurnik, Foster City, CA (US)

Correspondence Address: JONES DAY, 222 EAST 41ST ST, NEW YORK, NY, 10017, US

(21) Appl. No.: 11/326,803

(22) Filed: Jan. 6, 2006

Related U.S. Application Data
(63) Continuation of application No. 10/330,314, filed on Dec. 27, 2002, now abandoned.

(51) Int. Cl. C07K 1/26; G01N 27/447

(52) U.S. Cl. 204/453; 204/604

(57) ABSTRACT

A microfluidics device and method for sample loading, concentrating, mixing, and/or reacting is disclosed. The device has a microchannel network that includes a channel segment communicating with first and second reservoirs. A projection formed on a wall portion of the channel segment terminates therein at a point or edge. When a voltage potential is applied across the two reservoirs, the projection functions to create an electric field gradient within the channel segment that causes charged components in the channel segment to concentrate in the region of the projection. The device is useful, for example, in loading a sample of dilute charged components for electrophoretic separation in the device.

### FIELD OF THE INVENTION

The field of this invention is microfluidic devices and, in particular, a device designed for improved sample handling operations, such as sample loading, concentrating, mixing and reacting.

### BACKGROUND OF THE INVENSION

Microtechnology has already and continues to revolutionize numerous aspects of performing operations. As part of this revolution, microfluidics offers small compact devices to perform chemical and physical operations with minute volumes. In this manner, numerous events may be simultaneously performed within a small area using orders of magnitude less reagent and sample than possible with conventional 96-well plates.

One aspect of microfluidics is the use of capillary electrokinetics to move materials in small volumes from one site to another within closed channels created in a solid substrate. Referred to commonly as μTAS or “lab-on-a-chip,” these devices offer numerous advantages for performing chemical operations. The devices allow for mixing, carrying out chemical reactions, such as the polymerase chain reaction, genetic analysis, screening of physiological activity of drug candidates, and diagnostics, to mention only the more popular applications. The devices permit the use of much smaller amounts of reagents and sample, permit faster reactions, allow for easy transfer from one reaction vessel to another and separation of charged entities for rapid and accurate detection.

Numerous designs have been described in the literature for performing these operations in conjunction with particular protocols. Generally, one has a plurality of intersecting channels, particularly channels which join at an intersection. By applying appropriate voltage gradients, the volume in which the ions of interest reside can be relatively sharply delineated within a small volume, referred to as a plug. However, the limited volume of the sample plug can limit the total molar amount of sample components that can be loaded. For dilute sample components, this may lead to poor resolution or inability to detect sample components present only at low concentrations. Although the total sample loading volume can be increased, e.g., in a double-T type channel configuration, sample volumes may not stack well prior to electrophoretic separation, leading to poor resolution between peaks, and in any case, total available loading volume may be limited by space constraints in a microfluidics device.

It would thus be desirable to provide a microchannel device and method that allows for efficient loading of dilute-component samples in a relatively small loading volume. Such a device and method would have applications in several sample-handling operations, including sample loading, concentrating, mixing, and reacting.

### SUMMARY OF THE INVENTION

In one aspect, the invention includes a microfluidics device for use in handling a sample that contains charged components. The device has a substrate having a microchannel network formed in the substrate, e.g., within a covered surface region of the substrate. The network includes a channel segment defined by a channel-forming wall portion. The segment communicates with first and second reservoirs, which have or are adapted to receive first and second electrodes, respectively, by which a voltage potential can be applied between the reservoirs.

According to an important feature of the device, the channel segment contains a projection that extends from the wall portion into an interior region of the segment, terminating therein at a point, edge, or surface. The projection functions to create an electric field gradient within the channel segment, when a voltage potential is applied across the channel, between the first and second reservoirs, that causes charged components in a sample added to the first reservoir, or between the first reservoir and the projection, to concentrate in the region of the projection.

In various embodiments, the projection has a triangular or rectangular shape in a longitudinal cross-section, and/or an arcuate edge in a transverse cross-section. The channel segment is preferably between 0.1 μm to 1 mm deep, 0.5 μm to 2 mm wide, has a cross-sectional area between 0.1 μm² to about 0.25 mm². The projection preferably extends into the interior of the channel segment a distance at least about 10%, typically 10-30%, of the channel width.

In one embodiment, e.g., for use in electrophoretic separation of loaded sample components, the microchannel network includes a main sample-handling channel and first and second side channels that intersect the main channel at axially spaced first and second ports, respectively, where the channel segment is the portion of the main channel between and including the ports. The first and second side channels have distal ends that communicate with the first and second reservoirs, respectively, and the main channel has upstream and downstream ends that communicate with third and fourth reservoirs, respectively. Preferably, the intersection of the main channel and first side channel is formed by a rounded wall portion.

The device may further include a third side channel that terminates at a third reservoir and intersects the main channel at a third port disposed between the first port and said projection.

In another aspect, the invention includes a method for concentrating charged components in a sample. In the method, the sample is added to a microfluidics device of the type described above, i.e., a device having a channel network that includes a channel segment and first and second reservoirs communicating with the channel segment. After adding the sample, a voltage potential is applied between said first and second reservoirs, creating an electric field gradient within the channel segment. By means of a projection that extends from a wall portion of the channel segment into an interior region of the segment, and terminates therein at a point, edge, or surface, the electric field gradient within the channel segment is altered so as to cause charged components in the sample contained in the first reservoir, and between the first reservoir and the projection, to concentrate in the region of the projection.

For use in electrophoretically separating charged components in the sample, the channel segment may be a portion of a separation channel having upstream and downstream ends. Here the sample is added by placing it in the first reservoir and/or between the first reservoir and the projection. Application of a voltage potential between the first and second reservoirs is effective to move charged components in the sample in an upstream direction in the channel segment, toward the projection. The method further includes applying a voltage potential across the ends of the separation channel, to separate sample components concentrated in the region of the projection by electrophoretic movement of the components in a downstream direction within the separation channel.

In this embodiment, the channel network may include a first side channel that intersects the main channel at a first port and communicates with the first reservoir, said the sample-adding step may include adding the sample to the first reservoir. The channel network may further include a second side channel that intersects the main channel at a second port and communicates with the second reservoir, where the channel segment is the portion of the main channel between and including the ports. Applying the voltage potential is effective to move charged sample components in an upstream direction in the channel segment from the first port toward the second port.

For use in mixing charged components from two different samples, the channel network may include a first side channel that (i) intersects the main channel at a first port and (ii) communicates with said first reservoir, and an auxiliary side channel that (i) intersects the main channel at an auxiliary port disposed axially between the first port and the projection, and (ii) communicates with an auxiliary reservoir. The sample-addition step includes adding a first sample to the first reservoir and a second sample to the auxiliary reservoir. Applying a voltage potential between the first and second and between the auxiliary and second reservoirs, causes charged sample components from both samples to migrate toward and concentrate in the region of the projection.

More generally, the invention provides a method of concentrating charged species contained in a microfluidics channel at a selected region in the channel. The method is carried out by interposing adjacent the selected region, a projection that extends from a wall portion of the channel segment into an interior space thereof, and terminates therein at a point, edge, or surface, and applying a voltage potential across the channel.

These and other objects and features of the invention will become more fully apparent when the following detailed description of the invention is read in conjunction with the accompanying drawings.

### BRIEF DESCRIPTION OF THE DRAWINGS

FIG. 1 illustrates an embodiment of a microfluidics device of the invention, having a double-T sample-injection channel network, and shown with other components of a microfluidics systems for carrying out one method of the invention;

FIGS. 2A and 2B are alternative transverse sectional views taken along section line 2-2 in FIG. 1, illustrating a triangular projection formed on a wall surface of the substrate in the device (2A), and on the cover in the device (2B);

FIG. 3 is a transverse sectional view of the device taken along line 3-3 in FIG. 1;

FIGS. 4A and 4B are enlarged plan views of (4A) the sample-injection of the microchannel network indicated at 4A in FIG. 1, illustrating a triangular projection for field focusing, and (4B) the region indicated at 4B in FIG. 4A;

FIG. 5 is an enlarged plan view of a microchannel region like that shown in FIG. 4A, but illustrating a rectangular projection for field focusing;

FIG. 6 is an enlarged plan view of a microchannel region like that shown in FIG. 4A, but illustrating a circumferential triangular projection for field focusing;

FIGS. 7A-7C illustrate sample injection and separating steps in the embodiment of the device illustrated in FIGS. 1A and 1B;

FIG. 8 is an electropherogram showing maximum calculated concentrations of sample-component peaks produced by three sample injection and separation methods, including one using a triangular projection in the loading channel, in accordance with the invention;

FIGS. 9A and 9B are electropherograms showing maximum calculated concentrations of sample-component peaks produced by different channel and projection configurations;

FIGS. 10A-10C illustrate sample loading, mixing, reacting, and separating steps in accordance with another embodiment of the invention;

FIGS. 11A-11C illustrate sample loading and separating steps in accordance with a third embodiment of the invention; and

FIGS. 12A-12C illustrate sample loading and separation steps in accordance with a fourth embodiment of the invention.

### DETAILED DESCRIPTION OF THE INVENTION

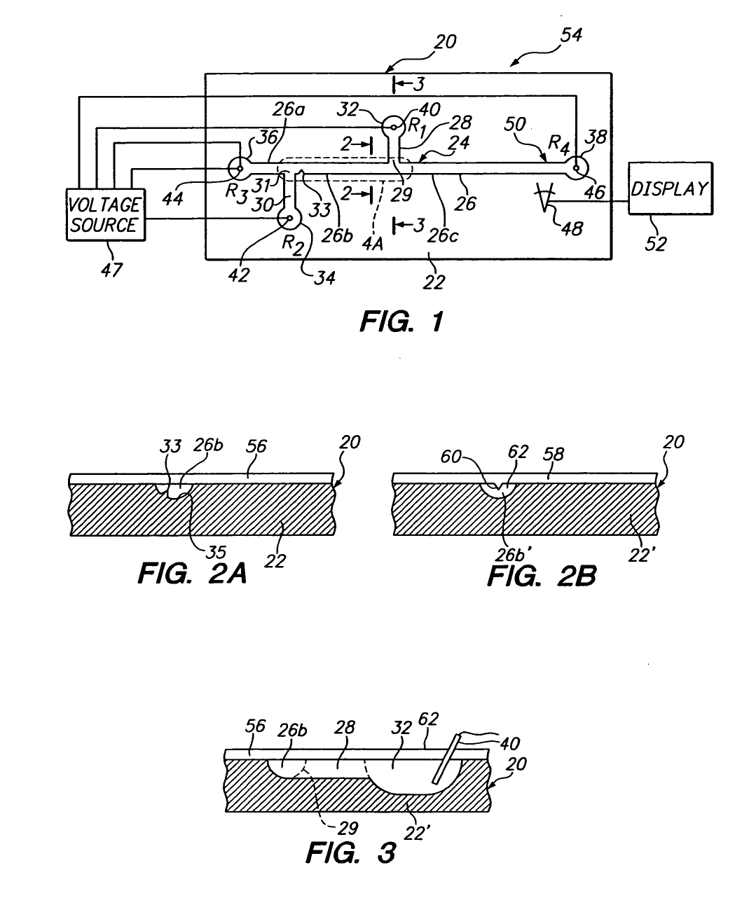

FIG. 1 illustrates a microfluidics device 20 constructed in accordance with one embodiment of the invention. The device includes a substrate 22, and a microchannel network 24 formed in the substrate. By “microchannel network” is meant one of more microchannels, hereafter referred to as channels, that are preferably between 0.1 μm to 1 mm deep, 0.5 μm to 2 mm wide, and have a cross-sectional area between 0.1 μm² to about 0.25 mm². The network in device 20 includes a main channel 26, a pair of side channels 28, 30, and first, second, third, and fourth reservoirs 32, 34, 36, 38, respectively, that communicate with the distal ends of the first and second side channels, and the upstream and downstream ends of the main channel, respectively.

As seen, side channels 28, 30, intersect the main channel at ports 29, 31, dividing the main channel into three regions: an upstream region 26a extending between reservoir 38 and port 31, a sample-loading region 26b extending between and including the ports 31, 29, and a separation region 26c downstream of port 29. The sample-loading region, also referred to herein as an offset, as typical dimensions between about 50-500 μM. As will be seen, the length of the offset may shift the electric field, and thus the observed electrophoretic mobility of a charged species loaded into and electrophoretically separated in the device. However, a significant advantage of the invention is that high resolution can be achieved with an offset in the range of less than 1 mm, and typically less than 500 μM, and may be as low as 50 μM or less.

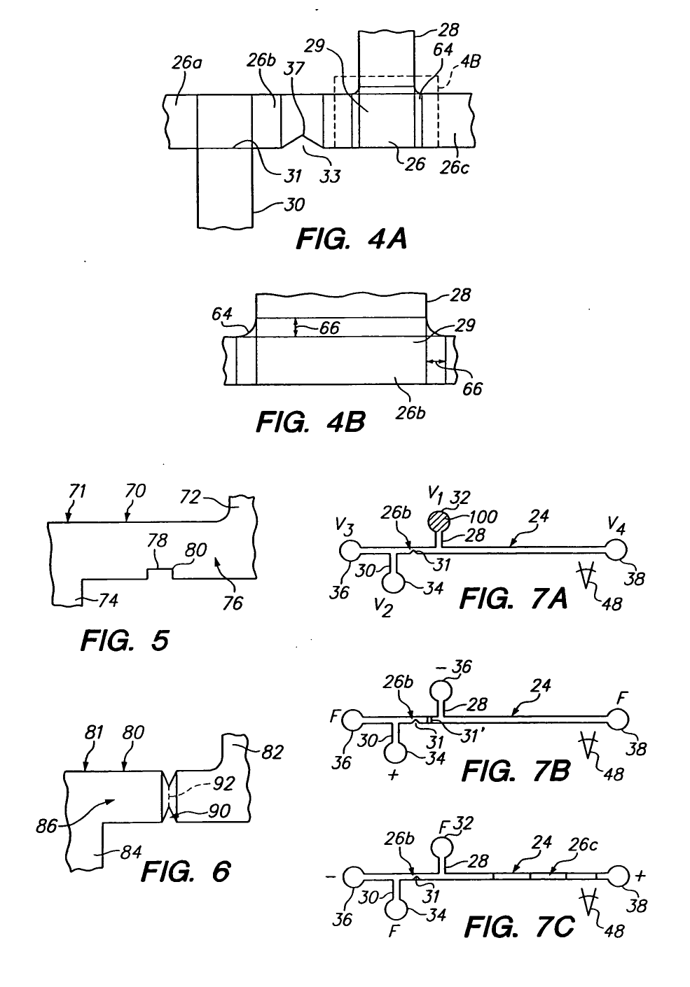

In accordance with an important feature of the invention, the sample-loading, or sample-injection region includes a projection 33 extending from a wall portion of the channel into an interior channel space, terminating at a point or an edge, as will be detailed below with reference to FIGS. 4-6.

Each reservoir provides, or is adapted to receive, an electrode, such as electrodes 40, 42, 44, and 46 in reservoirs 32, 34, 36, 38, respectively. The electrodes are operatively connected to a power source 47, as indicated, for applying a voltage potential across selected pairs or sets of electrodes, and thus across associated reservoirs in the device, when the reservoirs and channels in the network contain an electrolyte solution, e.g., an aqueous buffer solution. The power source may be a conventional DC voltage source capable of applying selected voltage potentials sufficient to achieve electric fields in the range 100-1,000 volts/cm over selected time periods, either to pairs to electrodes or simultaneously to more than two electrodes.

Also shown in the figures is a detector 48 used for detecting sample components, e.g., fluorescence-labeled components, as they pass through a detection zone 50 in the separation region of the main channel. The detector is operatively connected to a display 52 at which detector events, e.g., in the form of an electropherogram, can be displayed to the user. Collectively, the device, power source, detector and display form a microfluidics system 54 for carrying out various sample loading, concentrating, mixing, reacting, and/or separating steps, as well be considered below.

FIG. 2A, is a transverse cross-section view of the device taken along section line 2-2 in FIG. 1, i.e., in a section plane perpendicular to the axis of the separation channel. As seen here, the channel network, as represented by a portion of channel region 26b, is formed in substrate 22 and enclosed by a cover 56 which is attached by sealing to the upper surface of the substrate in the figure. In the embodiment shown here, projection 33 is formed on a wall portion 35 of the channel section, terminating at a point within the channel below the surface of the cover.

In another embodiment, illustrated in FIG. 2B, the channel network formed in substrate 22′ is enclosed by a cover 56 which provides an upper channel wall-forming portion 62 that carries a projection 60 that extends into the interior of the channel, and terminates at a point therein. In still another embodiment, not shown, the cover that encloses the channel network may be detachably placed over the substrate, allowing the channel network to be exposed, and/or the one or more additional covers to be substituted. For example, if it is desired to be able to place one or more projections, such as projection 60 in FIG. 2A, at different selected locations within a channel network during different, separate microfluidics operation, a first cover with one selected arrangement of projection(s) could be employed in one operation. This cover could then be replaced by a second cover having another arrangement of projection(s) for a second operation.

FIG. 3 is a transverse cross-sectional view of device 20 taken along line 3-3 in FIG. 1, that is, through reservoir 40 and along side channel 28. As seen, channel region 26b and side channel 28 have the depth dimensions, and are substantially shallower than reservoir 32 which preferably has a substantially greater volume capacity than the channels in the network. Also shown is electrode 40 received in reservoir 32 through cover 56, and an opening 62 in the cover by which liquid, e.g., sample, can be introduced into or withdrawn from the reservoir, for example, through a capillary tube placed through the opening.

In construction, the substrate or card in which the microchannel network is formed will generally have a thickness of at least about 20 μm, more usually at least about 40 μm, and not more than about 0.5 cm, usually not more than about 0.25 cm. The width of the substrate will be determined by the number of units (either separate channels in a single network or multiple discrete networks) to be accommodated and may be as small as about 2 mm and up to about 6 cm or more. The dimension in the other direction will generally be at least about 0.5 cm and not more than about 50 cm, usually not more than about 20 cm, and frequently not more than about 10 cm. An exemplary embodiment is roughly 8×12 cm, in conformity to the so-called “SSB Standard” dimensions of microtitre plates. The substrate may be a flexible film or relatively inflexible solid, where the microstructures, such as reservoirs and channels, may be provided by embossing, molding, machining, etc. The substrate may be of any convenient material, such as glass, plastic, silicon, fused silica, or the like, where depending on the nature of the operation, the channel surface may be coated to encourage or discourage or control the direction of electro-osmosis.

The capillary channels may vary as to dimensions, width, depth and cross-section, as well as shape, being rounded, trapezoidal, rectangular, etc. The path of the channels may be straight, rounded, serpentine, meet at corners, cross-intersect, meet at tees, or the like. Certain channel features related specifically to the present invention will be detailed below with reference to FIGS. 4-6. The channel dimensions will generally be in the range of about 0.1 μm to 1 mm deep and about 0.5 μm to 2 mm wide, where the cross-sectional area will generally be 0.1 μm² to about 0.25 mm². The channel lengths will vary widely depending on the operation for which the channel is to be used. The central separation channel will generally be in the range of about 0.05 mm to 50 cm, more usually in the range of about 0.5 mm to 10 cm, and in many cases not more than 5 cm, while the various portions of the channels other than the primary channels, the peripheral channels, will be within those ranges and frequently in the lower portion of the range.

The reservoirs will generally have volumes in the range of about 10 nl to 10 μl, usually having volumes in the range of about 20 nl to 4 μl. The reservoirs may be cylindrically shaped or conically shaped, particularly inverted cones, where the diameter of the open end or face of the reservoir will be from about 1.5 to 25 times, usually 1.5 to 15 times, the diameter of the bottom of the reservoir, where the reservoir connects to the channel.

Depending upon which layer serves as the channel layer, and the manner in which the channels are produced, e.g. embossed or molded, the enclosing surface will be below the channels to enclose them or above the channels to enclose them. When below, where for example the channels and reservoirs are molded into the substrate, an enclosing film or plate material may serve as a support for the device. Alternatively, the channels may be formed by embossing or molding, where the enclosing material is a cover. The substrate and/or the enclosing film may serve to form the reservoirs. The supporting film or plate material will generally be at least about 25 μm and not more than about 5 mm thick. The film or plate material used to enclose the channels and the bottom of the reservoirs will generally have a thickness in the range of about 10 μm to 2 mm, more usually in the range of about 20 μm to 1 mm. The selected thickness is primarily one of convenience and assurance of good sealing and the manner in which the devices will be used to accommodate instrumentation. Therefore, the ranges are not critical.

As indicated, the substrate may be a flexible film or inflexible solid, so the method of fabrication will vary with the nature of the substrate. For embossing, at least two films will be used, where the films may be drawn from rolls, one film embossed and the other film adhered to the embossed film to provide a physical support. The individual units may be scored, so as to be capable of being used separately, or the roll of devices retained intact. See, for example, application serial no. PCT/98/21869. Where the devices are fabricated individually, they will usually be molded, using conventional molding techniques. The substrates and accompanying film will generally be plastic, particularly organic polymers, where the polymers include addition polymers, such as acrylates, methacrylates, polyolefins, polystyrene, etc. or condensation polymers, such as polyethers, polyesters, e.g. polycarbonates, polyamides, polyimides, polysiloxanes, etc. Desirably, the polymers will have low fluorescence inherently or can be made so by additives or bleaching. The underlying enclosing film will then be adhered to a substrate by any convenient means, such as thermal bonding, adhesives, etc. The literature has many examples of adhering such films, see, for example, U.S. Pat. Nos. 4,558,333; and 5,500,071.

FIG. 4A is an enlarged plan view of the portion of the channel network indicated at 4A in FIG. 1, where like structures are indicated with like numerals. The figure shows, in particular, the relative shape and size of projection 33, terminating at a point 37 within channel region 26a between ports 29, 31, where side channes 28, 30, respectively, intersect main channel 26. In this figure, channel width is about 85 μm, and the projection, has base and height dimensions (the base and height of the triangle shown) of about 62 μm and 20 μm, respectively. More generally, the projection extends into the channel a distance of at least 1% of the channel width, and typically 10-40% of the channel width. The cross-section shape of the projection in transverse cross-section may also be triangular, i.e., where the projection is a pyramidal structure. Alternatively, the projection may be in the form of an annular arc in transverse cross-section, defining an arcuate edge within the channel region. In another embodiment, the projection may terminate at a surface, rather than a point or edge. The end surface is spaced from the walls of the channel and separated therefrom by the sides of the projection. Also shown in the figure is the rounded wall portion 64 at the intersection of side channel 28 and main channel 26. This feature is seen in further enlargement in FIG. 4B, which shows the region where channel 28 intersects main channel 26 at port 29. The dimensions of the rounded wall portion in the figure, indicated at 66 in the figure, are about 10 μm in each direction, relative to a channel width of about 85 μm. The rounded wall portion at the channel intersection acts to reduce field concentration effects that would occur with a sharp edge-like intersection, such as shown for side channel 30 in FIG. 4A. Although the latter intersection could also be formed with a rounded wall portion, electric field effects will be less critical at this boundary, as will be seen below.

FIG. 5 is an enlarged plan view of a sample-loading region in a microfluidics device like that described above. The view corresponds approximately to that of FIG. 4A, showing a device 70 having a main channel 71, and first and second side channels 72, 74, respectively, that intersect the main channel and define therebetween, a sample-loading region 76. A projection 80 in the device has a rectangular cross section in planar cross-section, i.e., in a section plane containing the long axis of the channel, with exemplary width and height dimensions of about 60 μm and 20 μm, respectively. As above, the projection may have a triangular shape in transverse cross-section, in which case the projection forms upstream and downstream points, such as point 80. Alternatively, the projection may have an arcuate cross-section in transverse cross-section, defining upstream and downstream arcuate edges.

Yet another embodiment of a projection in the device is illustrated in FIG. 6, which corresponds approximately to FIG. 4A, showing a device 80 having a main channel 81, and first and second side channels 82, 84, respectively, that intersect the main channel and define therebetween, a sample-loading region 86. A projection 90 in the device has a triangular cross section in a planar cross-section, with exemplary width and height dimensions of about 20 μm and 20 μm, respectively. As indicated, the triangular projections extends around the entire substrate wall portion, defining an interior arcuate edge 92.

### EXEMPLARY EMBODIMENTS AND METHODS

FIGS. 7A-7C illustrate the use of device 20 in FIG. 1 in a method for sample injection and electrophoretic separation and identification of charged sample components. The channel network in an exemplary embodiment has an upstream channel length of 4 mm, a sample loading region or offset length of 250 μM, and a separation channel region length of 11 mm. Each of the side channels has a 4 mm length.

Initially, a sample, indicated by shading at 100 in FIG. 7A, is injected into sample (first) reservoir 32, with the remainder of the network being filled by a electrolyte solution, e.g., standard electrophoresis buffer. The sample typically contains one or a number of charged components, such as the electrophoretic tags described in co-owned patent applications are described in co-owned U.S. Patent Application for “Methods and Reagents for Catalytic Multiplexed Assays”, Ser. No. 09/293,821, filed May 26, 2001, incorporated by reference and attached hereto. The electrophoretic tags are generated in a multiplexed analyte-detection reaction in which a plurality of labeled probes, when interacting specifically with target molecules, are cleaved to release target-specific tags. Detection of specific targets can then be detected by electrophoretic separation and identification of the released tags. Often, one or more of the charged components in a sample will be present in very dilute concentrations, e.g., on the order of nM to fM concentration levels.

According to an important advantage of the invention, the device allows for sample concentration, substantially independent of offset length and volume, so that sample components present only at very dilute original concentrations can be readily detected and, optionally, quantitated. Additionally, there is no need to “pinch” the sample during the injection step, by simultaneously applying a voltage potential across V₃, V₄. This pinching effect, as is known, acts to shape a sample plug contained in the offset by creating buffer flow from opposite reservoirs of the main channel into the first and second side channels. Because the boundaries of the stacked plug in the present invention are spaced from the side-channel ports, there is no benefit in pinching.

After sample injection, a voltage potential is applied across reservoirs 32, 34 (V₁, V₂), with the other reservoirs allowed to have floating potentials. For purposes of this embodiment, it is assumed that the sample components of interest are negatively charged, and that V₂ has the higher voltage potential, e.g., V₂=500V, V₁=0 (ground). During this loading period, negatively charged sample components move electrophoretically from sample reservoir 32 toward reservoir 34, that is, through side channel 28 and upstream toward projection 31. In accordance with the invention, the distortion in the electric field produced by projection 31 causes charged components to accumulate and concentrate at a region 31′ adjacent the project, as indicated in FIG. 7B. Although some charged sample material may pass upstream beyond the projection and into reservoir 34, the overall effect of the loading is to produce a several-fold concentration of charged components at the stacking region, with longer loading times producing greater accumulation of components. In FIG. 14, the projection is shown as 33, whereas in FIGS. 7A,B,C it is shown as 31. Although the stacking region is indicated as just downstream of projection 31, a square or rectangular projection may produce stacking on either side of the upstream and downstream projection points or throughout the length of the projection.

Following this loading and concentrating step, the components in the sample can be separated electrophoretically, by applying an appropriate voltage potential across reservoirs 36, 38 (V₃, V₄), and allowing V, and V₂ to float. This step is referred to as sample separation. In accordance with the invention, the relative absence of sample components in side channel 30, and the severalfold higher concentration of sample components in the stacked sample plug, relative to the concentration of sample components in side channel 28, allows for electrophoretic movement and separation of the plug components in the separation channel without simultaneous “pull-back” of material into the side channels. Avoiding pull-back increases the amount of sample material that migrates into the separation region of the main channel by up to 50%, thus further improving the ability to detect low-concentration sample components.

As seen in FIG. 7C, the separation step ultimately results in electrophoretic separation of sample components. These components are detected by detector 48 as they pass through a detection zone, for generating a suitable display, e.g., electropherogram.

To demonstrate the advantages of the invention, the present invention was compared, by modeling, with a method carried out in a conventional microfluidics device (no field-distorting projection, and a sharp boundary between each side channel and the main channel). The latter method was modeled under conditions both with and without pinching and pull-back.

For both types of devices used in the example, the modeling conditions involved initially coating the channels with 1% polyethylene oxide (PEO), then filling with 25 mM HEPES buffer, pH 7.38. The sample reservoir was modeled to contain 1 μM fluorescein in 25 mM Hepes buffer containing 25 mM NaCl. The offset was 250 μm, with other channel dimensions as given above. For sample injection, modeled voltages of V₁=0, V₂=500 volts were employed, with V₃ and V₄ allowed to float, or for pinching, simultaneous application of voltages V₃₌₀, and V4=0 volts. For sample separation, modeled voltages of V₃=0, V₄=700 volts were employed, with V₁ and V₂ allowed to float, or for pull-back, with simultaneous application of voltages of V₁=V₂=380 volts. The loading time was 12 seconds.

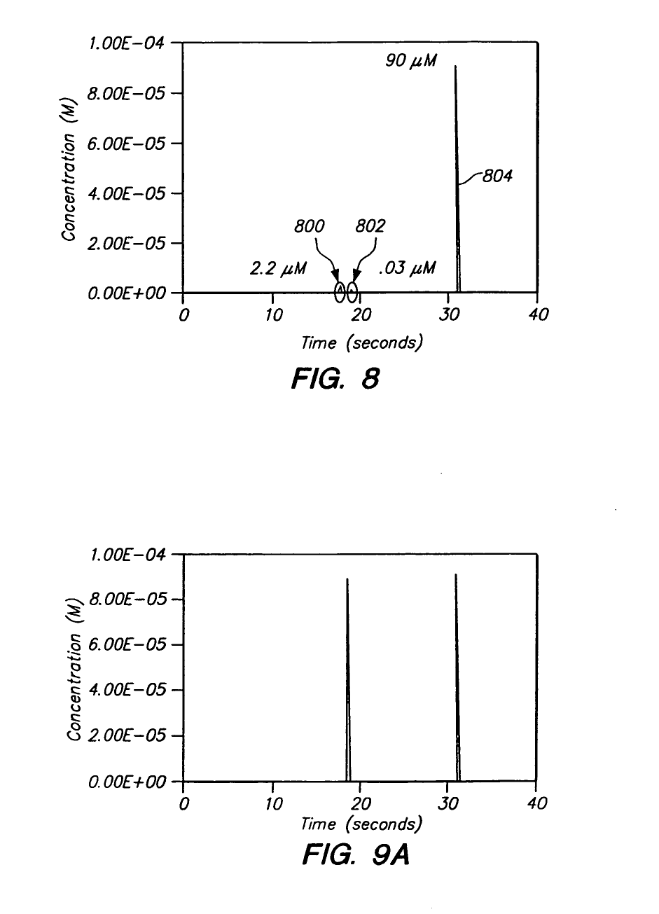

The resulting electropherograms for the three different modeled methods is shown in FIG. 8. As seen, the present invention gives a peak height corresponding to a concentration of 90 μM. Using instead a geometry without a triangular step gives a maximum concentration of 2.2 μM if floating electrodes are used, and 0.3 μM if pinch plus pullback is used. Thus, there is a 300×increase (90/0.3) in the sensitivity of the detector with this new geometry relative to that with pinch plus pullback.

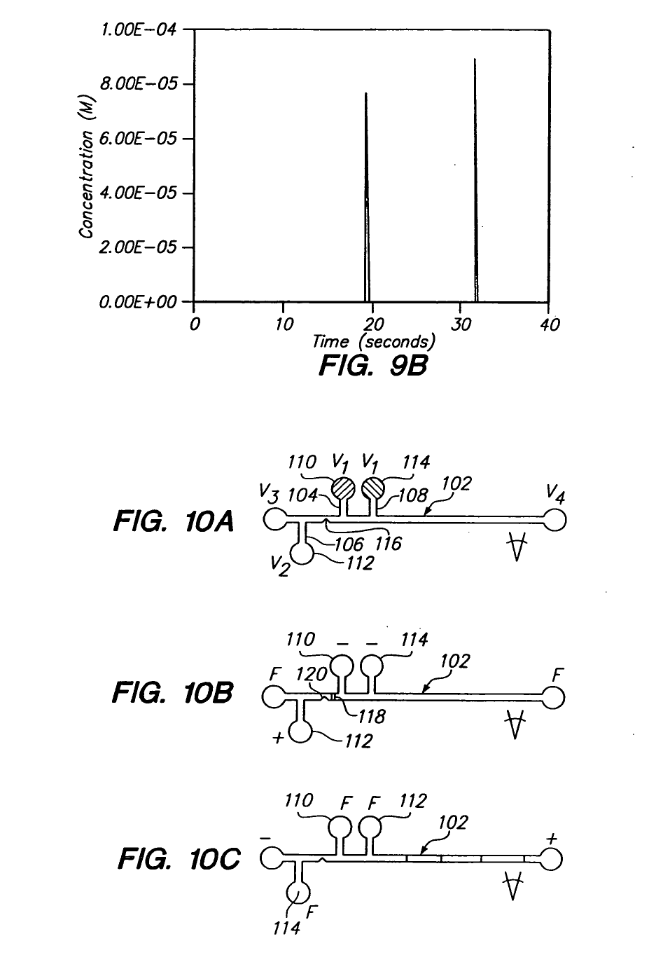

A similar modeled method was carried out, to compare the resolution in a device having a rectangular projection as illustrated in FIG. 5 with that having a triangular projection, under the same loading and injecting conditions described above. FIG. 9A shows the resulting modeled electropherogram comparing a square step of width and height 20 microns to the triangular step. It is seen that the maximum peak height of the square step is nearly identical to that of the triangular step (14% less), due largely to increased peak tailing.

Another comparison was modeled with a 20×20 micron step, but with a 500 micron offset versus the current 250 micron offset. The resultant electropherogram, showing the square step with a long offset to the triangular step with the short offset is shown in FIG. 8B. It is seen that the height of the peak is between that of the square with the short offset and the triangle with the short offset. This may be due to decreased tailing with a longer offset.

Thus, the method and device of the invention are effective to provide up to 100 fold of more increase in sensitivity, at the same time, avoiding pinch and pull-back during loading and injecting, respectively.

FIGS. 10A-10C illustrate device and method for mixing and concentrating, or mixing, concentrating and reacting, two different reagent solutions, prior to sample-component separation. The device employed here has a channel network 102 which is intersected by first and second side channels 104, 106, respectively, as above, and a third, auxiliary side channel 108. Side channels 104, 106, 108 terminate in reservoirs 110, 112, and 114, respectively.

Initially, the device is loaded with a first sample placed in reservoir 110, and a second sample or reaction reagent placed in auxiliary reservoir 114. When a voltage is placed across reservoirs 110, 112 (V₁), at one voltage, and reservoir 112 (V₂) at another voltage, with V₃ and V₄ allowed to float, as illustrated in FIG. 10B, charged sample and reagent material in the two upper reservoirs is drawn electrophoretically into a stacked plug 118 adjacent projection 120. As indicated above, the stacked plug may be on either side of 120, or throughout the offset region in the main channel. In this concentrated condition, charged components from the two reservoirs are intimately mixed. If the components are intended to react, reaction will occur at least to some extent, in the concentrated condition.

For example, the charged material in reservoir 110 may contain a sample of target polynucleotide sequences, and the charged material in reservoir 114, electrophoretic probes that can hybridize to the target sequences, with release of target-specific electrophoretic tags, under suitable reaction conditions. The latter, such as enzymic or non-enzymic cleaving agents, can be included in the bulk phase microfluidics buffer, or, if charged reagents, in one of the reservoirs. Alternatively, if the cleaving reaction requires an external stimulus, e.g., photolytic light, such stimulus can be applied when the two species have concentrated. In another embodiment, the charged material in one reservoir may be an enzyme, and the other reservoir, charged substrate electrophoretic probes which, when brought into contact with the probes, release substrate-specific electrophoretic tags.

After concentrating, mixing and (optionally) reacting the components in the sample plug, the device is switched to its separation mode, by applying a suitable voltage potential across V₃, V₄ and allowing V₁, V₂ to float, as shown in FIG. 1C. The charged components, which may include, for example, released electrophoretic tags, are then separated electrophoretically as shown.

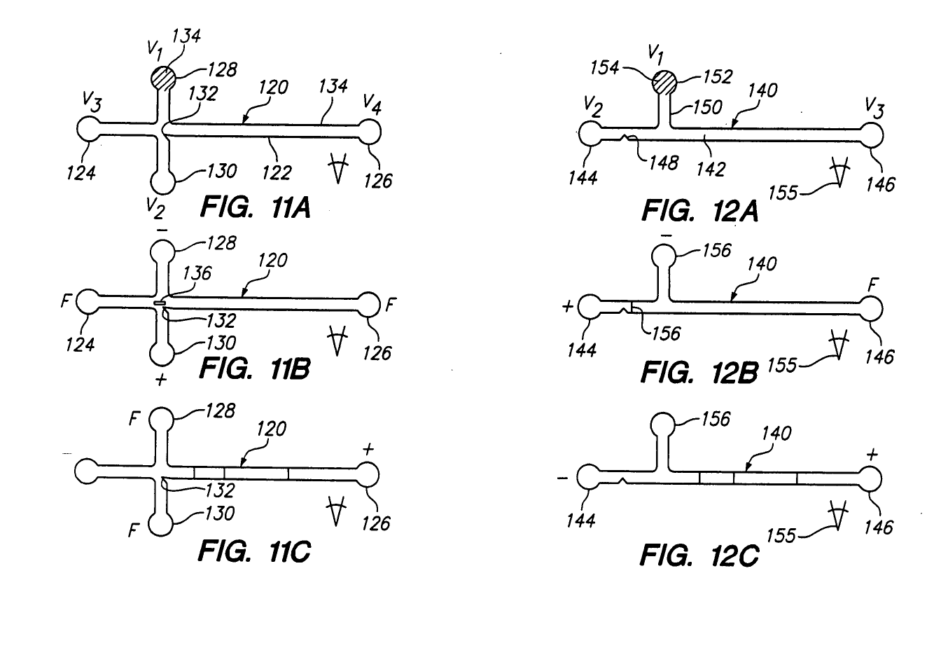

FIGS. 11A-11C illustrate an embodiment of the device, for use in concentrating charged sample components in one channel, for transfer of the stacked sample plug to another channel in a channel network. The channel network in the device, indicated at 120 includes a sample-supply channel 122 that terminates at reservoirs 124, 126, and a sample-receiving channel 128 that that intersects the first channel and terminates at its opposite end in a reservoir 130. The network includes a projection 132 just upstream of the intersection of the two channels. It will be appreciated that the network shown may be part of a more elaborate network, in which the two channels shown function to concentrate and transfer charged components from one network region to another.

In operation, channel 122 initially (or in the course of a microfluidics operation) contains a sample 134 of charged components that are to be transfer into side channel 128. To carry out this operation, a voltage is applied across V₁, V₂, as indicated in FIG. 11B, with V₃ floating. During this first step, charged sample components concentrate to form a stacked plug 136 adjacent projection 132. In the second step, a voltage is applied across V₁, V₃, as indicated in FIG. 11C, with V₂ floating, causing the stacked sample to migrate into channel 128, e.g., for removing the components, for separating the components, or for bringing the components into a second reaction zone.

FIGS. 12A-12C illustrate an embodiment of the invention that functions like device 20, but with only a single side channel. Specifically, the channel network in the device, indicated at 140 includes a main channel 142 that terminates at upstream and downstream reservoirs 144, 146, respectively, and a single side channel 150 that intersects the channel 142 terminates at its opposite end in a sample reservoir 152. The network includes a projection 148 just upstream of the intersection of the two channels.

In operation, sample 154 containing charged components is added to reservoir 152, with the remainder of the network filled with a suitable electrolyte, as above. For sample injection, a voltage potential is applied across reservoirs 152, 144 (V₁, V₂), with reservoir 146 allowed to float, as in FIG. 12B. The charged components in the sample concentrate in a stacked plug 156 adjacent projection 148, as shown in FIG. 12B. As above, it is noted that for a rectangular projection, the stacking region could be on either side of or throughout the projection region >>Following this loading, a voltage potential is applied across reservoirs 144, 146, as indicated in FIG. 12C, to separated sample components in the stacked band along the separation channel.

From the foregoing, it can be seen how various objects and features of the invention are met. The invention allows for the stacking of charged components at at selected region which can be easily engineered in a microfluidics device. The stacking allows for concentration, mixing, reacting, or stacking to occur at localized regions within a microfluidics device. This feature is particularly useful for sample stacking of dilute sample components prior to electrophoretic separation of the components.

Although the invention has been described with respect to certain embodiments and applications, it will be appreciated that various changes and modifications can be made without departing from the invention.

What is claimed is:

1. A microfluidics device for use in handling a sample that contains charged components, comprising
a substrate,
formed in the substrate, a microchannel network that includes a channel segment communicating with first and second reservoirs, said segment being defined by a channel-forming wall portion, and said reservoirs having or being adapted to receive first and second electrodes, respectively, by which a voltage potential can be applied across the reservoirs, and
means defining a projection that extends from said wall portion into an interior space in the segment, terminating therein at a point, edge, or surface, whereby a voltage potential applied between the first and second reservoirs creates an electric field gradient within the channel segment that causes charged components in a sample added to the first reservoir, or between the first reservoir and the projection, to concentrate in the region of the projection

2 The device of claim 1 wherein said projection has a triangular or rectangular shape in a longitudinal cross-section.

3. The device of claim 1, wherein said projection has an arcuate edge in a transverse cross-section.

4. The device of claim 1, wherein said microchannel network is formed in a surface region of the substrate, the device further includes a cover sealed against a surface of the substrate, enclosing the microchannel network, and said projection is formed on said cover for projecting into an interior space in said channel segment.

5. The device of claim 1, wherein said channel segment is between 0.1 μm to 1 mm deep, 0.5 μm to 2 mm wide, has a cross-sectional area between 0.1 μm² to about 0.25 mm², and said projection extends into the interior of the channel segment a distance at least about 10% of the channel width.

6. The device of claim 1, wherein (i) said microchannel network includes a main sample-handling channel and first and second side channels that intersect the main channel at axially spaced first and second ports, respectively, (ii) said channel segment is the portion of the main channel disposed between and including said ports, (iii) said first and second side channels have distal ends that communicate with said first and second reservoirs, respectively, and (iv) the main channel has upstream and downstream ends that communicate with third and fourth reservoirs, respectively.

7. The device of claim 6, wherein the intersection of said main channel and first side channel is formed by a rounded wall portion.

8. The device of claim 6, which further includes an auxiliary side channel that terminates at an auxiliary reservoir and intersects the main channel at an auxiliary port disposed between the first port and said projection.

9. A method of concentrating charged components in a sample, comprising
adding the sample to a microfluidics device that includes a channel network having a channel segment and first and second reservoirs communicating with the channel segment,
applying a voltage potential between said first and second reservoirs, thereby creating an electric field gradient within the channel segment, and
by means of a projection that extends from a wall portion of the channel segment into an interior space of the segment, and terminates therein at a point, edge, or surface, altering the electric field gradient within the channel segment to cause charged components in the sample added to the first reservoir, or between the first reservoir and the projection, to concentrate in the region of the projection.

10. The method of claim 9, wherein the projection has a triangular or rectangular shape in a longitudinal cross-section.

11. The method of claim 9, wherein said projection has an arcuate edge in a transverse cross-section.

12. The method of claim 9, wherein said channel segment is between 0.1 μm to 1 mm deep, 0.5 μm to 2 mm wide, has a cross-sectional area between 0.1 μm² to about 0.25 mm², and said projection extends into the interior of the channel segment a distance at least about 10% of the channel width.

13. The method of claim 9, for use in electrophoretically separating charged components in a sample, wherein said channel segment is a portion of a separation channel having upstream and downstream ends, said channel network includes a first side channel that intersects the main channel at a first port and communicates with said first reservoir, said adding includes placing said sample in said first reservoir and/or between the first reservoir and said projection, said applying is effective to move charged components in said sample in an upstream direction in said channel segment, toward said projection, and the method further includes applying a voltage potential across the ends of the separation channel, to separate sample components concentrated in the region of the projection by electrophoretic movement of the components in a downstream direction within the separation channel.

14. The method of claim 13, wherein said channel network includes a second side channel that intersects the main channel at a second port and communicates with said second reservoir, said channel segment is between and includes said first and second ports, and said applying is effective to move charged sample components in an upstream direction in said channel segment from said first port toward said second port.

15. The method of claim 9, for mixing charged components from two different samples, wherein said channel network includes a first side channel that (i) intersects the main channel at a first port and (ii) communicates with said first reservoir, and an auxiliary side channel that (i) intersects the main channel at an auxiliary port disposed axially between said first port and said projection, and (ii) communicates with an auxiliary reservoir, said adding includes adding a first sample to the first reservoir and a second sample to the auxiliary reservoir, and said applying includes applying a voltage potential between the first and second and auxiliary and second reservoirs, causing charged sample components from both samples to migrate toward and concentrate in the region of the projection.

16. A method of concentrating charged species contained in a microfluidics channel at a selected region in the channel, comprising
interposing adjacent the selected region, a projection that extends from a wall portion of the channel segment into an interior space thereof, and terminates therein at a point or edge, and
applying a voltage potential across the channel.

17. The method of claim 16, wherein the projection has a triangular or rectangular shape in an longitudinal cross-section.

18. The method of claim 16, wherein said projection has an arcuate edge in a transverse cross-section.

19. The method of claim 16, wherein said channel segment is between 0.1 μm to 1 mm deep, 0.5 μm to 2 mm wide, has a cross-sectional area between 0.1 μm² to about 0.25 mm², and said projection extends into the interior of the channel segment a distance at least about 10% of the channel width.
